(19) United States
(12) Patent Application Publication
(10) Pub. No.: US 2002/0016545 A1
(43) Pub. Date: Feb. 7, 2002

(54) Mobile ultrasound diagnostic instrument and system using wireless video transmission

(76) Inventors: Jens U. Quistgaard, Seattle, WA (US); Blake W. Little, Bothell, WA (US)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/833,499

(22) Filed: Apr. 11, 2001

Related U.S. Application Data
(60) Provisional application No. 60/197,358, filed on Apr. 13, 2000.

(51) Int. Cl. A61B 8/00

(52) U.S. Cl. 600/437; 600/446

(57) ABSTRACT

An ultrasound diagnostic system includes a battery powered mobile instrument which includes an ultrasound transducer for transmitting and receiving ultrasonic waves and signal processing circuitry for processing the received ultrasound waves and formatting video signals which drive a modulator which encodes the video signals as RF analog waveforms by wireless transmission. A remote console unit receives the transmitted RF analog signals and further processes the video signals for display on a monitor. Thus, the processing and display capabilities of a larger remote console is combined with the mobile diagnostic instrument for enhanced signal processing and diagnostics.

### CROSS-REFERENCES TO RELATED APPLICATIONS

This patent application claims priority from U.S. Provisional Application No. 60/197,358, filed Apr. 13, 2000.

### BACKGROUND OF THE INVENTION

This invention relates generally to medical ultrasonic diagnostic systems, and more particularly the invention relates to a mobile ultrasound diagnostic instrument which is operable with physically removed monitoring apparatus.

Modern ultrasonic diagnostic systems are large, complex instruments. Today's premium ultrasound systems, while mounted in carts for portability, continue to weigh several hundred pounds. In the past, ultrasound systems such as the ADR 4000 ultrasound system produced by Advanced Technology Laboratories, Inc. were smaller desk top units about the size of a personal computer. However, such instruments lack many of the advanced features of today's premium ultrasound systems such as color Doppler imaging and three dimensional display capabilities. As ultrasound systems become more sophisticated they also become bulkier.

Traditional medical ultrasonic imaging devices produce video images which are typically displayed on a cathode ray tube (CRT) device, though some more modern devices utilize flat panel displays such as liquid crystal displays. These displays are generally integral to the ultrasound imaging device, and are typically used to present information in a standard broadcast format, e.g. NTSC or PAL video. In addition, traditional devices typically provide an analog video output which enables connection of external video devices such as video printers, video recorders, external video monitors, and video distribution or digitization systems which may be connected for remote viewing or storage systems.

To this point, such display and video interconnect capabilities have been sufficient in the medical ultrasound community because ultrasound imaging equipment has typically been bulky and therefore not highly portable. For this reason, use patterns have not developed where physical interconnection of video devices is particularly inconvenient. Typically, video devices are either placed on a mobile cart with the ultrasonic imaging device and are semi-permanently physically connected to the ultrasonic imaging device, or these devices are not used when the ultrasonic imaging device is used in a portable fashion. Despite the foregoing, however, there are situations in traditional ultrasound imaging where it would be advantageous to alleviate or eliminate the requirement of physically interconnecting video peripheral or network devices in a simple and cost-effective manner.

Disclosed in U.S. Pat. No. 5,722,412 is a diagnostic ultrasound instrument which exhibits many of the features of a premium ultrasound system in a hand-held unit. The instrument can be produced as a single unit or in a preferred embodiment the instrument is a two-part unit one including a transducer, beamformer, and image processor and the other including a display and power source for both units. In such a configuration the transducer/processor unit can be manipulated with one hand with a cable between the two units enables the video to be shown on the display unit while the lateral unit is held or positioned for optimal viewing of the ultrasound image. The cable also provides energy for the transducer/processor unit from the display unit.

The advent of highly portable ultrasound imaging devices (HPUIDs), such as disclosed in U.S. Pat. No. 5,722,412, supra, alters the situation previously described. HPUIDs typically weigh less than ten pounds and are battery powered, thus enabling use patterns that are significantly different from traditional scenarios. For example, HPUIDs enable physicians to conveniently carry an ultrasound imaging system from room to room in a hospital or clinic setting. This highly portable use model is, however, compromised by the necessity to carry external video devices such as printers with the HPUID, and/or have such devices physically connected to the HPUID. Thus it is desirable to implement technology that alleviates or eliminates this problem in a simple and cost-effective manner.

Disclosed in copending application Ser. No. 60/132,515 filed May 4, 1999 is a mobile ultrasound diagnostic instrument such as disclosed in the '412 patent and a docking stand which facilitates independent use of the instrument for diagnostic purposes or use of the instrument with larger, more complex systems. The mobile ultrasound diagnostic instrument includes a docking stand for a self-powered ultrasound console. The console includes electronics for driving a transducer array and processing reflected ultrasound waves, and a visual display for processed ultrasound waves. The docking stand includes a sleeve for slidably receiving the console of the instrument, the sleeve being configured to expose the visual display and manual controls of the console. A vertical support positions the sleeve in a raised position above a base for the vertical support. The sleeve also includes a signal connector for mating with a connector of the console and receiving video signals for auxiliary display. The video signals are transmitted through a cable from the console to auxiliary monitoring apparatus.

In accordance with the principles of the present invention, a diagnostic ultrasound instrument is provided which incorporates a mechanism for wireless transmission and reception of analog video signals to enable interaction with external video devices.

### SUMMARY OF THE INVENTION

In accordance with the invention, an ultrasound diagnostic system is provided in which transducer means transmits and receives ultrasonic waves and signal processing means processes the ultrasonic waves and formats video signals for display. Modulation means encodes the video signals and modulates an RF carrier signal which is then transmitted wirelessly to a remote device for further processing and display.

In a preferred embodiment, the ultrasound system also includes a receiver which can receive processed signals from the remote device for display.

In a preferred embodiment, the ultrasound system is a highly portable ultrasound imaging device weighing less than ten pounds with the transducer and signal processing circuitry being battery powered. Video signals are then transmitted to an external remote device having enhanced signal processing capability for further signal processing and display. The wireless transmission and reception of analog video signals permit enhanced instrument operation by enabling the use of remote equipment through a wireless communication channel without the need for a cable.

The invention and objects and features thereof will be more readily apparent from the following detailed description and independent claims when taken with the drawing.

### BRIEF DESCRIPTION OF THE DRAWINGS

FIG. 1 is a functional block diagram of a portable ultrasound diagnostic apparatus wirelessly communicating with a remote video processing and display system in accordance with the invention.

FIG. 2 is a functional block diagram of the portable ultrasound device of FIG. 1 in accordance with one embodiment of the invention.

FIG. 3 is a more detailed functional block diagram of the portable ultrasound device of FIG. 2.

### DETAILED DESCRIPTION OF THE ILLUSTRATED EMBODIMENTS

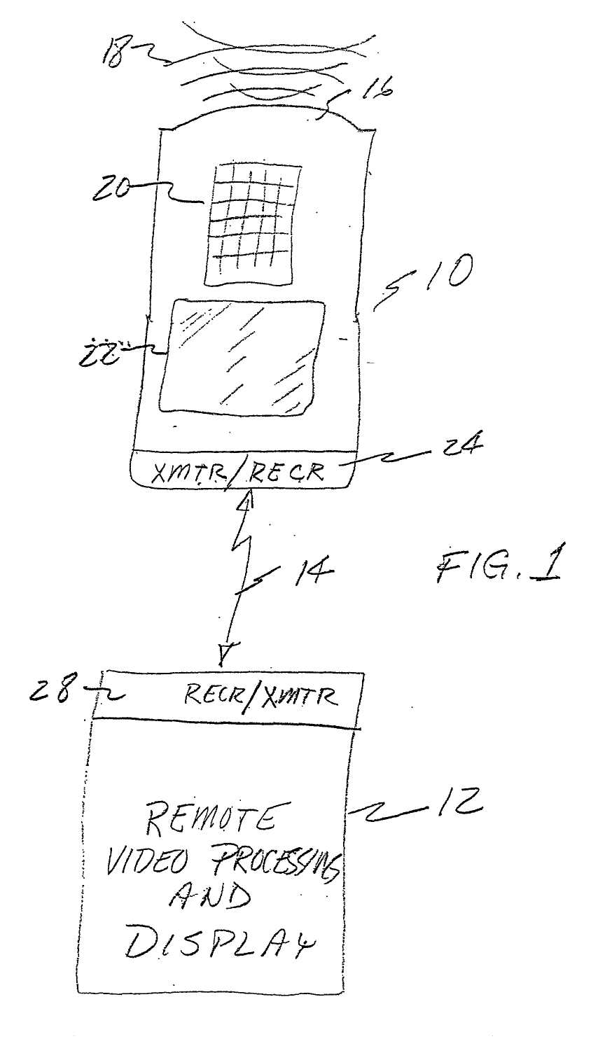

FIG. 1 illustrates a portable ultrasound diagnostic device shown generally at 10 which communicates with a remote video processing and display unit shown generally at 12 through a wireless link 14. The portable device 10 is preferably of the type disclosed in U.S. Pat. No. 5,722,412 which is battery powered for ease of operation. Device 10 includes a transducer 16 for transmitting and receiving ultrasonic waves 18 and a manual control panel 20 for controlling operation of the device. A liquid crystal display or other suitable display 22 is provided on the portable unit for the display of video signals. Signal processing circuitry within unit 10 (not shown) processes the ultrasonic signals from transducer 16 and provides video signals for display.

In accordance with a feature of the invention, portable unit 10 includes a RF transmitter/receiver 24 which encodes the video signals from the signal processing circuitry and modulates an RF carrier signal to provide an RF analog waveform which can be transmitted at 14 to a receiver 28 of the remote processing and display unit 12. Unit 12 can be a larger immobile console unit with enhanced signal processing capability and larger display. Thus, a physician can operate the remote unit in an examination room with signals being transmitted to the console unit located in another unit of a hospital for processing and display.

In accordance with another feature of the invention, the processed signal from console unit 12 can be transmitted back to the portable unit 10 for reception and display on the display 22 of the portable unit. Thus, the utility of the small portable unit 10 weighing less than ten pounds and as little as five pounds is enhanced by employing the remote processing and display unit through the wireless link 14.

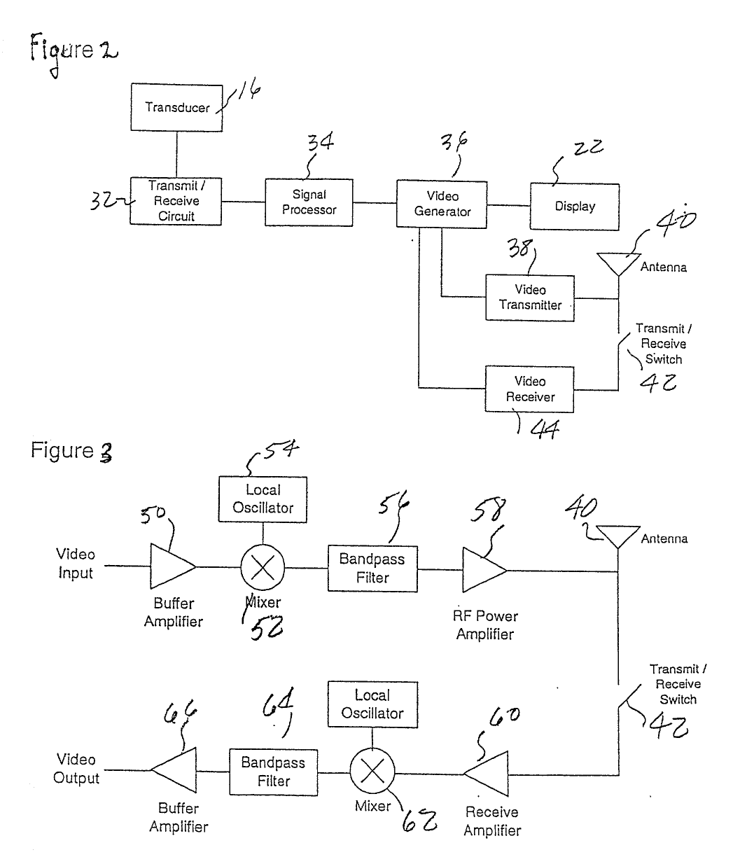

FIGS. 2 and 3 are more detailed functional block diagrams of the portable unit 10 of FIG. 1. In FIG. 2 an array transducer unit 16 transmits and receives ultrasonic waves under control of the circuitry 32 which converts ultrasonic waves to electrical waves and vice versa. Circuitry 32 includes a beamformer which can be analog, digital, or hybrid analog/sample data beamformer. The electrical signal from circuit 32 is then passed to a signal processor 34 for formatting video signals preferably in a standard broadcast format such as NTSC or PAL format. The formatted signals from video generator 36 can then be used to drive display 22 in the portable unit and in accordance with the invention are passed to a video transmitter 38 which modulates an RF carrier signal with the video signals for transmission from an antenna 40.

A transmit/receive switch 42 is provided so that RF signals from the remote console can be received by receiver 44 and then passed to the video generator 36 for control of display 22. Thus, the processing capability of the console is imparted to the portable unit for enhanced signal processing and display.

FIG. 3 is a more detailed functional block diagram of video transmitter 38 and video receiver 44 of FIG. 2. The video transmitter 38 includes a buffer amplifier 50 which applies the video signals to a mixer 52 that amplitude modulates the RF carrier signal from a local oscillator 54. The modulated carrier signal is then band-passed filtered at 56 and applied to an RF power amplifier 58 for driving the antenna 40. The video receiver 44 (FIG. 2) includes a receive amplifier 60 with the RF carrier signal from the amplifier 60 being demodulated by mixer 62 with the recovered video signal then passed through a band pass filter 64 and buffer amplifier 66 as a video output signal.

The use of wireless video transmission in accordance with the invention enhances the diagnostic capabilities of a small mobile ultrasound diagnostic instrument by allowing the processing capability and display of a remote console to be used therewith. While the invention has been described with reference to specific embodiments, the description is illustrative of the invention and is not to be considered as limiting the invention. For example, other known signal modulation can be employed. Thus, various modifications and applications may occur to those skilled in the art without departing from the true spirit and scope of the invention as defined by the appended claims.

What is claimed is:

1. A mobile ultrasound diagnostic instrument comprising
transducer means for transmitting and receiving ultrasonic waves,
signal processing means for processing received ultrasonic waves and formatting video signals for display,
modulator means for encoding the video signals as RF analog waveforms, and
transmission means for a wireless transmission of the analog waveforms to a receiver of a remote device for further processing and display.

2. The portable ultrasonic diagnostic device as defined by claim 1 wherein the modulator performs an amplitude modulation of the video signals on an RF carrier signal.

3. The mobile ultrasound diagnostic instrument as defined by claim 2 and further including a video display for displaying video signals.

4. The mobile ultrasound diagnostic instrument as defined by claim 3 and further including a video receiver for receiving processed signals from the console unit for display on the video display.

5. The mobile ultrasound diagnostic instrument as defined by claim 4 wherein the transducer means includes an array transducer and beam formation circuitry.

6. The mobile ultrasound diagnostic instrument as defined by claim 5 wherein the beam formation circuitry comprises an analog beamformer.

7. The mobile ultrasound diagnostic instrument as defined by claim 5 wherein the beam formation circuitry is a sampled data beamformer.

8. The mobile ultrasound diagnostic instrument as defined by claim 5 wherein the beam formation circuitry comprises a digital beamformer.

9. The mobile ultrasound diagnostic instrument as defined by claim 5 wherein the beam formation circuitry comprises a hybrid analog/sampled data beamformer.

10. The mobile ultrasound diagnostic instrument as defined by claim 5 wherein the total weight of the mobile instrument is less than fifteen pounds.

11. The mobile ultrasound diagnostic instrument as defined by claim 10 wherein the mobile instrument weighs less than ten pounds.

12. The mobile ultrasound diagnostic instrument as defined by claim 10 wherein the mobile instrument weighs less than five pounds.

13. An ultrasound diagnostic system comprising
a) a mobile instrument including
transducer means for transmitting and receiving ultrasonic waves,
signal processing means for processing received ultrasonic waves and formatting video signals for display,
modulator means for encoding the video signals as RF analog waveforms, and
transmission means for a wireless transmission of the analog waveforms to a receiver of a remote device for further processing and display, and
b) a console unit remote from the mobile instrument and including
a receiver for receiving RF analog waveforms transmitted by the mobile instrument,
video processing circuitry for further processing of the transmitted RF waveforms, and a display for displaying the processed video signals.

14. The ultrasound diagnostic system as defined by claim 13 wherein the remote console includes a transmitter for transmitting processed video signals back to the mobile instrument for display.

15. The ultrasound system as defined by clam 13 wherein the modulator of the mobile instrument performs an amplitude modulation of an RF carrier signal with the video signals.

16. The mobile ultrasound diagnostic instrument as defined by claim 13 wherein the transducer means includes an array transducer and beam formation circuitry.

17. The mobile ultrasound diagnostic instrument as defined by claim 13 wherein the beam formation circuitry comprises an analog beamformer.

18. The mobile ultrasound diagnostic instrument as defined by claim 13 wherein the beam formation circuitry is a sample data beamformer.

19. The mobile ultrasound diagnostic instrument as defined by claim 13 wherein the beam formation circuitry comprises a digital beamformer.

20. The mobile ultrasound diagnostic instrument as defined by claim 13 wherein the beam formation circuitry comprises a hybrid analog/sample data beamformer.

21. The mobile ultrasound diagnostic instrument as defined by claim 13 wherein the total weight of the mobile instrument is less than fifteen pounds.

22. The mobile ultrasound diagnostic instrument as defined by claim 13 wherein the mobile instrument weighs less than ten pounds.

23. The mobile ultrasound diagnostic instrument as defined by claim 13 wherein the mobile instrument weighs less than five pounds.
